(19) United States
(12) Patent Application Publication
(10) Pub. No.: US 2020/0155599 A1
(43) Pub. Date: May 21, 2020

(54) ANTI-EGFR/HIGH AFFINITY NK-CELLS COMPOSITIONS AND METHODS FOR CHORDOMA TREATMENT

(71) Applicant: NantKwest, Inc., San Diego, CA (US)

(72) Inventors: Patrick Soon-Shiong, Los Angeles, CA (US); John Lee, Topanga, CA (US)

(73) Assignee: NantKwest, Inc., San Diego, CA (US)

(21) Appl. No.: 16/604,341

(22) PCT Filed: May 11, 2018
(86) PCT No.: PCT/US2018/032281

§ 371 Date: Oct. 10, 2019

Related U.S. Application Data
(60) Provisional application No. 62/504,689, filed on May 11, 2017.

(51) Int. Cl. A61K 35/17; A61K 39/395; A61P 35/00

(52) U.S. Cl. CPC A61K 35/17; A61P 35/00; A61K 39/39558

(57) ABSTRACT

Chordoma is treated in a patient by co-administration of an anti-EGFR antibody and high affinity NK cells (haNK). Most preferably, the antibody is non-covalently bound to a high affinity variant of a CD16 receptor or administered before transfusion of the haNK cells to so target the chordoma cells for cytotoxic cell killing by the haNK cells.

This application claims priority to copending U.S. Provisional Application with the Ser. No. 62/504,689, which was filed May 11, 2017.

### FIELD OF THE INVENTION

The field of the invention is modified immune competent cells for the treatment of diseases, especially as it relates to high affinity natural killer (haNK) cells and anti-EGFR compositions for treatment of chordoma.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Chordoma is a rare bone tumor and is thought to be derived from the residual notochord. Accounting for 20% of primary spinal tumors (1-4% of all malignant bone tumors), about 300 new cases per year are diagnosed in the United States, with approximately 2400 patients alive with chordoma in the U.S. The median overall survival from time of diagnosis is an estimated 6-7 years. Surgery followed by radiation therapy is the usual “standard of care,” but the anatomic location and size of the tumor often prevent curative excision with clear margins. Thus, relapse is common and metastases have been reported in up to 40% of cases. No agent has been approved by the U.S. Food and Drug Administration for chordoma therapy since it is largely resistant to standard cytotoxic chemotherapy, creating an urgent need for novel therapeutic modalities for chordoma.

More recently, new treatment regimens have been proposed based on various molecular profiles of chordoma. For example, miRNA was proposed to downregulate EGFR as described in PLoS One (2014), 9(3): e91546. Combined inhibition of IFG-1R and EGFR showed durable response in one trial (Front Oncol (2016); 6:98), while various small molecule inhibitors of EGFR such as erlotinib, gefitinib, lapatinib, sapitinib, or afatinib were described as potential therapeutic agents based on in vitro data in J Pathol (2016); 239: 320-334.

In other examples, an immune therapeutic approach targeting PD-L1 expressed on chordoma cells using avelumab (anti-PD-L1 antibody) was employed as described in Oncotarget (2016); 7(23):33498-511. While conceptually elegant, various difficulties nevertheless remain. Among other things, PD-L1 is also expressed on various non-chordoma cells and as such off-target ADCC may occur. Moreover, even under in vitro condition using IFN-gamma stimulation and normal donor NK cells, avelumab mediated ADCC was relatively low (about 25-35% lysis of all targeted chordoma cells). In still further known approaches, chordoma cells lines were irradiated in vitro with low dose ionizing radiation to increase EGFR expression and were then exposed to cetuximab (anti-EGFR antibody). Subsequent exposure to normal donor NK cells indicated some ADCC (see Abstract FASEB Journal, Vol. 31, No. 1 Suppl; Abstract No. 934.12: Exploiting Immunogenic Modulation in Chordoma: Sublethal Radiation Increases EGFR Expression and Sensitizes Tumor Cells to Cetuximab). However, radiation is often not well tolerated and ADCC activity without radiation was less than desirable. Therefore, most of the more recent attempts to treat chordoma were less than successful or have not resulted in a regimen approved by regulatory agencies.

Thus, while various treatment methods and compositions for chordoma are known in the art, all or almost all of them suffer from one or more disadvantages. Thus, there remains a need for improved compositions and methods for treatment of chordoma.

### SUMMARY OF THE INVENTION

The inventive subject matter is directed to compositions, kits, and methods of treatment of chordoma that includes co-administration of haNK cells with an anti-EGFR antibody to so trigger ADCC (antibody dependent cell-mediated cytotoxicity) and augment EGFR-based treatments. Most notably, therapeutic effect is attained not by way of interference with EGFR signaling, but via NK cell (and especially high-affinity NK cell) mediated cytotoxic cell killing. Thus, suitable anti-EGFR antibodies may be agonistic or antagonistic, or may elicit no signaling change in response to binding, and preferred NK cells will have a CD16 variant with a binding affinity to the Fc portion on an IgG that is above the affinity of a wild type CD16 (e.g., 158FF).

Therefore, in one aspect of the inventive subject matter, the inventor contemplates a method of treating chordoma that includes a step of co-administering an anti-EGFR antibody and a high affinity NK (haNK) cell to a patient in need thereof at a dosage effective to treat the chordoma. Most preferably, it is contemplated that the anti-EGFR antibody is a monoclonal antibody with binding specificity against human EGFR, and/or that the anti-EGFR antibody is an IgG1 to so trigger ADCC. Therefore, viewed from a different perspective, it is contemplated that the anti-EGFR antibody may be a humanized non-human anti-EGFR antibody, and most preferably is cetuximab.

With respect to administration it is generally contemplated that the anti-EGFR antibody is administered at a dosage of between 100 mg/m² and 1,000 mg/m², preferably at the same time as the haNK cell. Thus, the anti-EGFR antibody may also be bound to a high-affinity CD16 that is expressed on a surface of the haNK cell. Contemplated haNK cells are preferably administered at a dosage of between 5×10⁵ cells/kg and 5×10⁸ cells/kg, and it is further preferred that the haNK cells are a NK92 derivative and/or (typically intracellularly) express recombinant IL2. Moreover, it is generally preferred that the haNK cell is genetically engineered to have a reduced expression of at least one inhibitory receptor and/or that the haNK cell is genetically engineered to express a CD16 158V variant.

Where desired, contemplated methods may further include a step of administering a further cancer treatment to the patient, most typically an immune therapy (e.g., administration of a recombinant yeast or recombinant virus expressing a patient- and tumor-specific neoepitope, or administration of a recombinant yeast or recombinant virus expressing brachyury) and/or chemotherapy (e.g., administration of irinotecan, gemcitabine, capecitabine, 5-FU, FOLFIRI, FOLFOX, and/or oxiplatin). Moreover, suitable further cancer treatments may also comprise radiotherapy.

Therefore, the inventor also contemplates a pharmaceutical composition that includes an anti-EGFR antibody that is coupled to a high affinity variant of CD16, wherein the CD16 high affinity variant is expressed on the surface of a genetically engineered NK cell. With respect to the antibody and the genetically engineered cell, the same considerations as above apply. In addition, it is generally preferred that the pharmaceutical compositions will be formulated for transfusion and comprise between 1×10⁶ cells and 5×10⁹ cells.

Therefore, the inventors also contemplate a pharmaceutical kit that comprises an anti-EGFR antibody and a plurality of a high affinity NK (haNK) cells. Once more, with respect to the antibody and the genetically engineered cell, the same considerations as above apply. In view of the above, it should therefore be recognized that the inventors also contemplate the use of a high affinity NK (haNK) cell to augment treatment of chordoma wherein the treatment comprises administration of an anti-EGFR antibody.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1A is a schematic illustration of a treatment based on anti-EGFR and haNK cells.

FIG. 1B is a table listing frequencies of allelic variants and binding affinity for CD16 Fc receptors in human donor cells and in genetically engineered haNK cells.

FIG. 2 depicts various graphs for selected phenotypes of CD16 polymorphism-genotyped NK cells and haNK cells.

FIG. 3 is a graph depicting exemplary results for EGFR expression in selected chordoma cell lines.

FIG. 4 is a graphical representation of exemplary results for in vitro assays for ADCC activity mediated by cetuximab relative to an isotype control antibody.

FIG. 5 is a graphical representation of exemplary results for in vitro assays for ADCC activity mediated by cetuximab using FCGR3A (CD16 gene)-genotyped normal donor NK cells that expressed the FcgRIIIa (CD16)-158 FF, VF, or VV allele.

FIG. 6 is a graphical representation of exemplary results in which cetuximab increased haNK-cell lysis via ADCC in selected chordoma cell lines at two different time points indicating multiple cell killing by haNK cells.

FIG. 7 is a graphical representation of exemplary results for affinity of cetuximab to CD16 of selected NK cells versus haNK cells.

FIG. 8 is an exemplary treatment schema for Induction Phase as contemplated herein.

FIG. 9 is an exemplary treatment schema for Maintenance Phase as contemplated herein.

### DETAILED DESCRIPTION

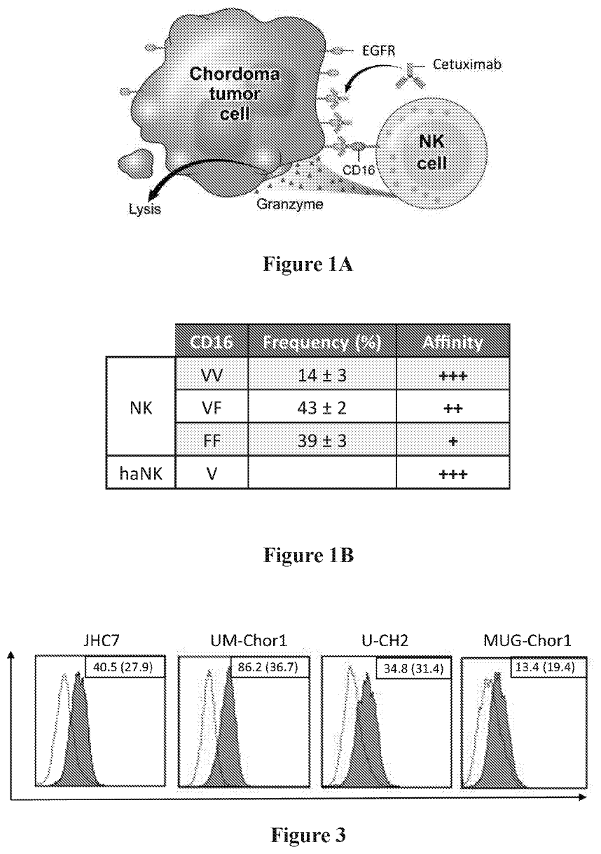

The inventor has now discovered that chordoma can be effectively treated using haNK cells in combination with an anti-EGFR antibody (e.g., cetuximab) to so induce in a patient an ADCC response/NK cytotoxic cell killing with desirable therapeutic effect as is exemplarily depicted in FIG. 1A. Such treatment may be implemented prior to, and/or concurrent with radio- and/or chemotherapy, and/or may be employed with immune therapy as is discussed in more detail below.

It should be noted that the antibodies contemplated herein are not used as an EGFR signaling inhibitor, but as an target specific beacon for a natural killer cell, and most preferably a high-affinity NK cell (haNK) to facilitate binding of the CD16 receptor of the NK cell to the Fc portion of the bound antibody and so to eradicate the tumor cell via ADCC/NK cytotoxic cell killing. With respect to the high affinity cells it should be appreciated that the high affinity may be due to patient idiosyncratic mutations at the CD16 locus (which may be hetero- or homozygous and occur at relatively low frequency), and more typically may be due to genetic engineering of NK cells to express a high affinity variant (e.g., F158V) from a recombinant nucleic acid. Therefore, it is typically preferred that the treatment includes combined administration of an anti-EGFR antibody and high affinity NK cells. Such administration may be performed sequentially, with the antibody being administered in a first step and the NK cells being transfused in a second subsequent step (e.g., within 24 hours of administration of the antibody), or simultaneously where the anti-EGFR antibody is bound to the CD16 receptor of the high affinity NK cell.

Therefore, in one preferred example, the inventor now contemplates that chordoma treatment with an anti-EGFR antibody can be significantly improved by co-administration of the anti-EGFR antibody with a genetically modified NK cell that expresses a high affinity CD16 variant (and where the NK cell most preferably also expresses intracellularly IL-2). Notably, due to the high affinity of the CD16 variant to the constant region of the antibody, tight binding and activation of the NK cell is achieved using the binding specificity of the anti-EGFR antibody to the EGFR of the tumor cell. Thus, it should be noted that contemplated treatments advantageously compensate for the most common, low affinity, variants of CD16 that is present in a large proportion of human (at least 70%). FIG. 1B depicts allele frequencies for CD16. Viewed from a different perspective, use of genetically modified NK cells will allow for an increase in ADCC in patients even where the patients have a low affinity CD16 (158F/F) phenotype. On the other hand, it is also contemplated that patients may also be identified as having a high-affinity CD16 (158V/V) phenotype. Such patients may then receive the anti-EGFR antibody without, or with a lower total dosage of haNK cells (e.g., between 10⁴-10⁶ cells or between 10⁵-10⁷ cells per transfusion).

With respect to suitable anti-EGFR antibodies it is contemplated that such antibodies may vary considerably in origin, sequence, and serotype. However, it is generally preferred that the anti-EGFR antibody will have a constant region (Fc) that binds with high affinity to the CD16 variant. Thus, and most typically, the constant region is a constant region of a human IgG₁ and the CD16 variant is a 158V/V variant. However, it should be appreciated that suitable CD16 variants and constant region variants may be specifically tailored to the specific antibody and/or a specific subset of genetically modified NK cells. As will be readily appreciated, high affinity pairs (CD16 variant/constant region variant) can be identified using numerous manners known in the art, and especially preferred manners include affinity maturation via phage display, RNA display, two-hybrid library screening using CD16 variant as bait and constant region library as prey (or vice versa), etc. Likewise, known high-affinity antibodies may be subject to CDR grafting (with the CDRs being specific towards EGFR) to so obtain a high-affinity anti-EGFR antibody.

Moreover, it should be recognized that while commercially available EGFR antibodies such as cetuximab and panitumumab are especially preferred, other contemplated anti-EGFR antibodies include monoclonal antibodies with binding specificity against human EGFR, and especially IgG₁ type antibodies that are humanized non-human anti-EGFR antibodies. There are numerous commercially available anti-EGFR antibodies known in the art (e.g., from ABCAM, Millipore, Biolegend, etc.), and all of them are deemed suitable for use herein. Additionally, suitable anti-EGFR antibodies may also include EGFR binding fragments that are coupled (preferably covalently as chimeric protein) to a CD16 binding domain (or domain variant).

For example, suitable anti-EGFR antibodies include clinically approved cetuximab and panitumumab, as well as human and non-human antibodies such as ab52894, ab131498, ab231, ab32562, ab32077, or ab76153 (all commercially available from Abcam, USA), as well as AY13 (Biolegend, USA) and 06-847 (Millipore, USA). These antibodies may be used directly, or in humanized form, or CDR regions may be grafted onto a human IgG. Likewise, suitable CDRs for grafting can be found in US584409 and WO 2011/156617.

With respect to suitable NK cells it is generally contemplated that the NK cells may be autologous NK cells from the patient, and such autologous NK cells may be isolated from whole blood, or cultivated from precursor or stem cells using methods known in the art. Moreover, it should be appreciated that the NK cells need not be autologous, but may also be allogenic or heterologous NK cells. Still further, it is contemplated that the NK cells may be HLA matched NK cells, which may be primary cells, NK cells differentiated from upstream stem or progenitor cells, or cultured NK cells. However, in particularly preferred aspects of the inventive subject matter, the NK cells are genetically engineered to achieve one or more desirable traits, and particularly preferred NK cells are NK92 cells, or derivatives of NK92 cells. Consequently, suitable NK cells will also be continuously growing (‘immortalized’) cells. For example, in one particularly preferred aspect of the inventive subject matter, the genetically engineered NK cell is a NK92 derivative that expresses IL-2 (typically in an intracellularly retained, non-secreted manner) and is modified to have reduced or abolished expression of at least one inhibitory receptor (KIR), which renders such cells constitutively activated (via lack of or reduced inhibition).

For example, suitable NK cells may have one or more modified KIR that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (‘activated natural killer cells).

In a particularly preferred aspect of the inventive subject matter, the NK cell is a genetically engineered NK92 derivative that is modified to express a high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to advantageously increase specific targeting and cytotoxic cell killing of tumor cells when using antibodies that are specific to a patient's tumor cells. Viewed from a different perspective, contemplated anti-EGFR antibodies will provide exquisite targeting specificity against chordoma cells while such genetically engineered NK92 derivative have high affinity to antibodies where the antibodies have bound to the cognate antigen, and further have significantly increased cytotoxic killing ability in the context of antibody binding. Of course, it should be appreciated that such targeting antibodies are commercially available and can be used in conjunction with the cells (e.g., bound to the Fcγ receptor). Likewise, such genetically engineered NK92 derivative cells may also be commercially obtained from NantKwest as haNK cells (‘high-affinity natural killer cells).

In further contemplated embodiments, the NK cells will be irradiated before transfusion to prevent continuous cell division. While not limiting to the inventive subject matter, the cells will typically be irradiated that abrogates cell division, but that still allows fort metabolic activity, and NK cell function (especially cytotoxic cell killing). Therefore, suitable radiation dosages for the NK cells will be between 50 cGy and 2,000 cGy. Furthermore, such radiation is typically beta or gamma radiation, however, other manners such as e-beam irradiation are also expressly contemplated herein.

Most typically, both the anti-EGFR antibody and the high affinity NK (haNK) cells are administered to the patient using dosages and routes that are known in the art for administration of both, antibodies and NK cells. Therefore, suitable dosages for administration of the anti-EGFR antibody (e.g., cetuximab) will typically be between 100 mg/m² and 1,000 mg/m², or between 100 mg/m² and 300 mg/m², or between 300 mg/m² and 600 mg/m², or between 600 mg/m² and 900 mg/m², or even higher. Administration is preferably intravenous over a period of between about 1 min and 120 min, and more typically between about 10 min and 60 min. Likewise, haNK cells are preferably administered at dosages suitable for cell transfusions. Therefore, suitable dosages will typically be in the range of between 5×10⁵ cells/kg and 5×10⁸ cells/kg, and most typically between 5×10⁶ cells/kg and 5×10⁷ cells/kg. Administration is preferably intravenous over a period of between about 1 min and 120 min, and more typically between about 10 min and 60 min.

In further contemplated aspects of the inventive subject matter, the administration of the anti-EGFR antibody and the haNK cells is preferably contemporaneous such that both the anti-EGFR antibody and the haNK cells are present in the patient's blood in measurable quantities at the same time. Consequently, co-administration of the anti-EGFR antibody and the haNK cells may be performed at the same time, or within 10 minutes or within 30 minutes or within 2 hours of each other. Moreover, it should also be appreciated that upon and/or during administration the anti-EGFR antibody may be non-covalently bound to the haNK cells via the CD16 variant.

Based on preclinical evidence of the role of EGFR in chordoma pathogenesis and the observation by immunohistochemistry that over 70% of chordoma specimens express EGFR, several clinical trials targeting EGFR have previously been undertaken in chordoma. However, because these trials were not randomized or well controlled, no consensus had been reached concerning the therapeutic benefit of EGFR inhibition in chordoma. In two separate case reports, the combination of the EGFR MAb cetuximab and gefitinib, a tyrosine kinase inhibitor of EGFR, achieved partial radiographically defined responses. Here, and as shown in more detail below, the inventor demonstrates that cetuximab, when combined with haNK cells, markedly and significantly increased NK cell based lysis, and especially lysis via ADCC.

Some previous clinical studies have also shown that FcgRIIIa polymorphisms of NK cells correlated with response to IgG₁ MAb therapy. Notably, patients with metastatic breast cancer who had FCGR2A-131 HH and/or FCGR3A-158 VV genotypes had a significantly better objective response rate and progression-free survival with trastuzumab therapy than patients with neither genotype. Similarly, in a study of 49 patients with follicular lymphoma, FCGR3A-158 VV patients had an improved response to rituximab. Three retrospective studies in metastatic colorectal cancer patients treated with cetuximab reported that VV is the most beneficial FCGR3A-158 genotype.

Although ADCC induction can be observed in in vitro models, clinical translation often raises various obstacles. First, recruiting sufficient numbers of functionally active NK cells to tumor tissues is technically challenging since they often represent only 10% of lymphocytes, and are frequently dysfunctional in a cancer-induced immunosuppressive environment. Moreover, first-line treatment for metastatic/advanced chordoma (i.e., chemotherapy and radiation therapy) is also very likely to reduce the number and activity of lymphocytes. Independently, adoptive NK-cell therapies have been developed to supply sufficient numbers of functional NK cells for patients. The cytotoxic NK-92 cell line was generated for adoptive transfer therapy from a 50-year-old male patient with progressive non-Hodgkin's lymphoma. Four phase I trials in different malignancies have been conducted using irradiated NK-92 cells. The infusions were well tolerated, and clinical responses were observed in patients with hematological malignancies, melanoma, lung cancer, and kidney cancers. However, since NK-92 cells do not express the FcgRIIIa receptor, they cannot mediate ADCC. In contrast, genetically engineered cells expressing a high-affinity CD16a. V158 FcγRIIIa receptor have now been established and are also commercially available (e.g., as haNK cells from NantKwest, 9920 Jefferson Blvd., Culver City, Calif. 90232).

Since only approximately 14% of the population is homozygous for the high-affinity FcgRIIIa receptor (FCGR3A-158 VV), the inventor contemplates infusing haNK cells into patients who carry the genotype of low- or intermediate-affinity FcgRIIIa receptor to maximize MAb efficacy. Among other things, and as shown in more detail below, the inventor noted that haNK cells have a 2.8-fold higher affinity to cetuximab than NK cells from healthy donors carrying FCGR3A-158 FF. Consistent with their high binding ability to cetuximab, haNK cells also significantly induced ADCC via cetuximab in chordoma cells. Moreover, since 10⁹ to 10¹⁰ irradiated NK-92 cells were shown to be safely administered to cancer patients, the inventor contemplates levels of adoptive transfer of irradiated haNK cells, even in patients whose endogenous NK cells express the VV phenotype (but possibly at a lower total dosage, such as 80% or less, or 70% or less, or 50% or less, or 40% or less than dosage administered to patient with 158FF phenotype).

NK-92 cells have been shown to express large numbers of activating receptors such as NKp30, NKp46, and NKG2D. NKG2D and DNAM-1 are the best-characterized activating NK-cell receptors implicated in immune response against cancers. Both receptors recognize their ligands expressed on tumor cells and induce target-cell lysis. As shown in more detail below, haNK cells have higher expression of NKG2D and DNAM-1 compared to normal NK cells, indicating a greater ability to recognize and lyse tumor cells. Notably, without cetuximab, NK cells from normal (158FF phenotype) donors lysed chordoma cells at extremely low levels without cetuximab (data not shown). In contrast, haNK cells induced substantially greater lysis of chordoma cells, even without cetuximab.

Consequently, it is contemplated that adaptively transferred irradiated haNK cells will provide sufficient numbers of functional NK cells for all chordoma patients and could so functionally ‘convert’ FCGR3A-158 FF carriers to VV carriers. Therefore, it should be appreciated that cetuximab plus irradiated haNK cell-mediated immunotherapy may have potential clinical benefit for patients with chordoma. Moreover, it should be recognized that while cetuximab is described as a suitable target, numerous additional or alternative targets are also deemed appropriate for use in conjunction with the teaching presented herein. For example, suitable targets include receptors and kinases that are preferably selectively or exclusively expressed at the cell surface of chordoma cells, and particularly include MET, PDGFR, and ERBB2. Moreover, where the chordoma cells have mutations that lead to neoepitopes in one or more proteins, it is contemplated that antibodies may be prepared that will bind to the neoepitope where the neoepitope is visible or presented on the surface of the cell.

Of course, it should be appreciated that additional therapeutic interventions may be used with or complement contemplated treatments. For example, suitable treatments include radiation and/or chemotherapy using agents such as irinotecan, gemcitabine, capecitabine, 5-FU, FOLFIRI, FOLFOX, and/or oxiplatin. In further contemplated aspects, contemplated treatments may also include immune modifiers such as IL15, IL15 superagonists, interferon-gamma to increase PD-L 1 expression, and/or checkpoint inhibitors targeting checkpoint receptors and/or their ligands (e.g., PD-L 1 antibody (avelumab)).

In addition, it is contemplated that immune therapy may also be based on generation of an immune response against brachyury. For example, immune therapy may be performed using recombinant viruses (and especially adenoviruses) that include a nucleic acid segment encoding brachyury (or a portion thereof). Infected cells, such as dendritic cells, will then express and process the recombinant protein for presentation as a MHC-I and/or MHC-II complex. In other aspects, heat-killed recombinant yeast may be genetically modified to express brachyury with potential antineoplastic activity. Upon subcutaneous administration, the brachyury-expressing yeast vaccine is then recognized by dendritic cells, processed, and presented by Class I and II MHC molecules on the dendritic cell surface, which is thought to elicit a targeted CD4+ and CD8+T-lymphocyte-mediated immune response.

### Examples

In Vitro Examples

Cell culture and reagents: The chordoma cell lines JHC7 and UM-Chor1 were obtained from the Chordoma Foundation (Durham, N.C.). The chordoma cell lines U-CH2 (ATCC® CRL-3218 TM) and MUG-Chor1 (ATCC® CRL-3219 TM) were obtained from American Type Culture Collection (Manassas, Va.). All cell lines were passaged for fewer than 6 months and were maintained as previously described (Oncotarget, 2016 May 9). haNK cells were cultured in phenol-red free and gentamycin-free X-Vivo-10 medium (Lonza, Walkersville, Md.) supplemented with 5% heat-inactivated human AB serum (Omega Scientific, Tarzana, Calif.) at a concentration of 5×10⁵/ml. haNK cells were irradiated with 10 Gy 24 h before all experiments. Peripheral blood mononuclear cells (PBMCs) from healthy volunteer donors were obtained from the NIH Clinical Center Blood Bank (NCT00001846).

Flow cytometry: Antihuman MAbs used were as follows: PE-EGFR (BD Biosciences, San Jose, Calif.), FITC-CD16 clone 3G8 (BD Biosciences), APC-CD56 (BioLegend, San Diego, Calif.), PE-CD226 (DNAM-1) (BD Biosciences), PerCP-Cy5.5-NKG2D (BD Biosciences), PE-Cy7-perforin (eBioscience, San Diego, Calif.). Samples were acquired on a FACSCalibur flow cytometer or FACSVerse (Becton Dickinson, Franklin Lakes, N.J.) and analyzed using FlowJo software (TreeStar, Inc., Ashland, Oreg.). Isotype control staining was <5% for all samples analyzed.

Antibodv-dependent cellular cytotoxicity assay: The ADCC assay was performed as known in the art, with indicated modifications. NK effector cells were isolated from normal donor PBMCs using the Human NK Cell Isolation (negative selection) Kit 130-092-657 (Miltenyi Biotec, San Diego, Calif.) following the manufacturer's protocol, resulting in >80% purity, and allowed to rest overnight in RPMI-1640 medium containing 10% fetal bovine serum. Tumor cells were harvested and labeled with ¹¹¹In. Cells were plated as targets at 2,000 cells/well in 96-well round-bottom culture plates and incubated with 10 μg/mL of cetuximab (Erbitux®; Lilly, Indianapolis, Ind.) or irresponsive rituximab (Rituxan®; Biogen, Cambridge, Mass.) as a control isotype antibody at room temperature for 30 min. NK cells or haNK cells were added as effector cells. Various effector:target cell ratios were used in the study. After 4 h or 20 h, supernatants were harvested and analyzed for the presence of ¹¹¹In using a WIZARD2 Automatic Gamma Counter (PerkinElmer, Waltham, Mass.). Spontaneous release was determined by incubating target cells without effector cells, and complete lysis was determined by incubation with 0.05% Triton X-100 (Sigma-Aldrich, St. Louis, Mo.). Experiments were carried out in triplicate. Specific ADCC lysis was determined using the following equation: Percent lysis=[(experimental cpm−spontaneous cpm)/(complete cpm−spontaneous cpm)]×100. To verify that CD16 (FcgRIII) on NK cells engages ADCC lysis mediated by cetuximab, a CD16 MAb was used to block CD16. NK cells were incubated with 2 μg/mL of CD16 MAb (clone B73.1; eBioscience) and haNK cells were incubated with 50 μg/mL of CD16 MAb for 2 h before being added to target cells.

CD16 (FcgRIIIa) genotyping: DNA was extracted from PBMCs of healthy donors using a QIAamp DNA Blood Mini Kit (Qiagen, Valencia, Calif.), and stored at −80° C. until use. The polymorphism of CD16 at amino acid position 158 that is a valine (V) vs. phenylalanine (F) was determined using allele-specific droplet digital polymerase chain reaction (PCR) employing the TaqMan array for CD16 (rs396991; Life Technologies, Waltham. Mass.). A master reaction mix was prepared, and 1 μL of genotyping DNA was added. The PCR reaction was performed on a Bio-Rad T100 thermal cycler (Bio-Rad, Hercules, Calif.) for 40 cycles at 95° C. for 10 min, 94° C. for 30 sec, and 60° C. for 1 min. The plate was read on a Bio-Rad QX200 droplet reader. Data were analyzed with Bio-Rad QuantaSoft v.1.5 software.

Statistical analysis: Significant differences in the distribution of data acquired by ADCC assays were determined by paired Student's t test with a 2-tailed distribution and reported as P values, using Prism 6.0f software (GraphPad Software Inc., La Jolla, Calif.).

The phenotype of CD16a polymorphism-genotyped NK cells and haNK cells: NK cells from some individuals can be potent cytotoxic effectors for cancer therapy. However, there can be technical challenges to obtaining sufficient numbers of functionally active NK cells from patients. As an alternative, several cytotoxic NK cell lines have been generated, including NK-92. These NK-92 cells, designated haNK, have recently been engineered to endogenously express IL-2 and the high affinity (ha) CD16 V158 FcγRIIIa receptor (haNK cells, commercially available from NantKwest, 9920 Jefferson Blvd., Culver City, Calif. 90232). The inventor compared the phenotype (CD56, DNAM-1, NKG2D, perforin, and CD16) of CD16a polymorphism-genotyped normal donor NK cells with that of haNK cells.

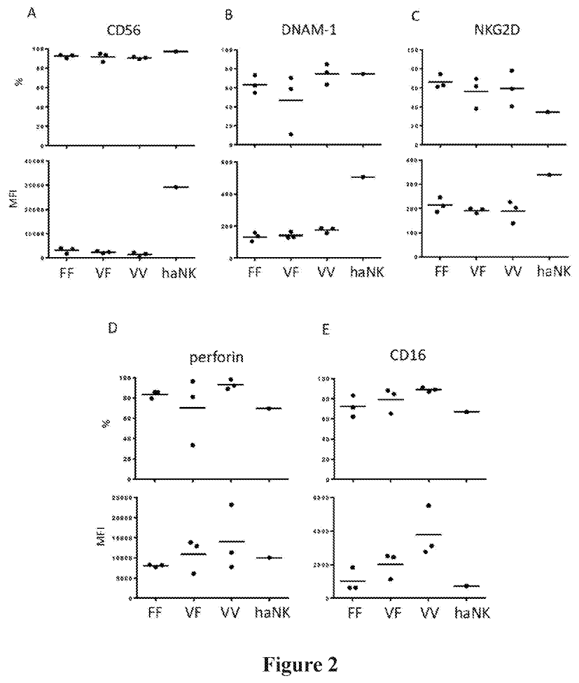

While there were only minor differences in the percentage of cells expressing a given marker, there were substantial differences observed in the levels of expression as determined by mean fluorescence intensity (MFI) as shown in the panels of FIG. 2. Compared to NK VV donors, haNK cells had a 20-fold higher MFI of CD56 (FIG. 2, Panel A), 2.9-fold higher expression of DNAM-1 (FIG. 2, Panel B), and 1.8-fold higher expression of NKG2D (FIG. 2. Panel C). Notably, there was no difference in perforin expression between NK cells and haNK cells (FIG. 2, Panel D), and the mean MFI of CD16 was 1.5-fold higher in VV donors compared to FF donors and haNK cells (FIG. 2. Panel E).

It has previously been shown that chordoma cell lines express EGFR, and the inventor qualitatively confirmed and extended this finding, employing four human chordoma cell lines: JHC7. UM-Chor1, U-CH2, and MUG-Chor1 with exemplary results shown in FIG. 3 (Inset numbers indicate % positive cells and mean fluorescence intensity (MFI)). As can be seen, the four chordoma cell lines express between 13% to 80% EGFR as determined by flow cytometry, although the absolute expression levels of EGFR can modulate with tissue culture density and time in culture.

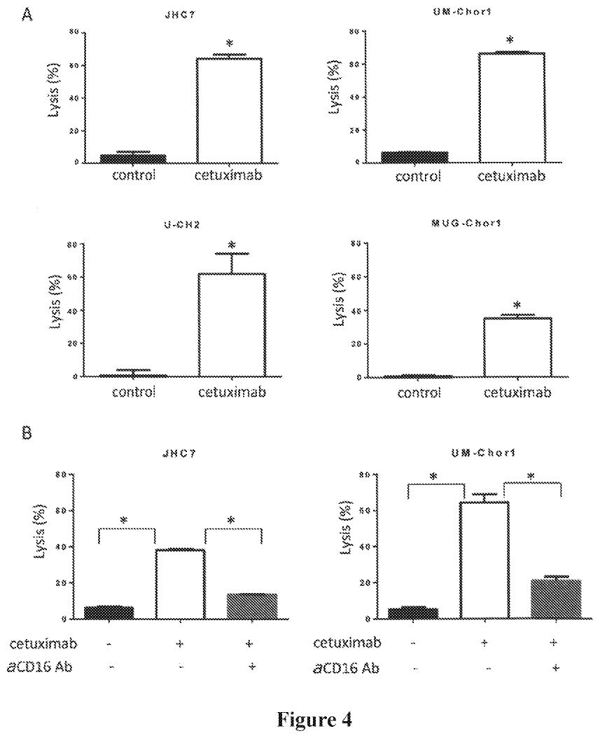

The inventor further performed an in vitro assay to determine cetuximab-mediated ADCC in chordoma cell lines employing NK cells from healthy donors as effectors. As shown in FIG. 4, Panel A, cetuximab significantly increased NK-cell lysis relative to the isotype control antibody in JHC7 cells (13.7-fold; P<0.01), UM-Chor1 cells (10.5-fold; P<0.01), U-CH2 cells (83.5-fold; P<0.01), and MUG-Chor1 cells (59-fold; P<0.01). Notably, cetuximab alone (no NK cells) did not mediate lysis of chordoma cells (data not shown). NK-cell lysis via ADCC occurs when CD16 (FcgRIII) on NK effector cells interacts with the Fc portion of antibodies recognizing target cells. As shown in FIG. 4, Panel B, the addition of CD16 neutralizing antibody inhibited cetuximab-enhanced NK-cell lysis in both the JHC7 and UM-Chor1 cell lines analyzed, indicating that cetuximab-induced NK-cell lysis was mediated by ADCC. More specifically, Panel A in FIG. 4 depicts results for ADCC assays for four chordoma cell lines, using normal donor NK cells at an effector:target (E:T) ratio of 20:1. Indicated groups were incubated with cetuximab. Panel B depicts results for ADCC assays with two chordoma cell lines, using normal donor NK cells at an E:T ratio of 20:1. Indicated groups were incubated with cetuximab and anti-CD16 antibody. Statistical analyses were done by Student's t-test, *=P<0.05, error bars indicate mean±S.D. for triplicate measurements. This experiment was repeated at least two times with similar results.

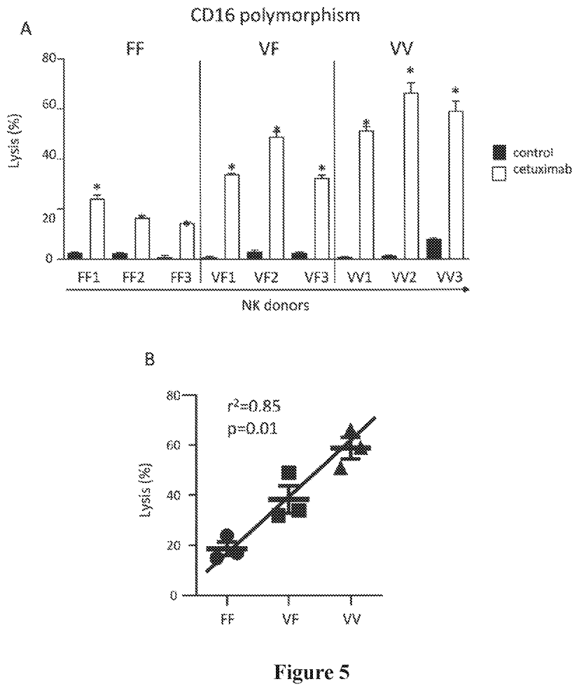

The inventor then performed in vitro assays for ADCC activity mediated by cetuximab using FCGR3A-genotyped normal donor NK cells that expressed the FcgRIIIa-158 FF, VF, or VV allele. With control isotype antibody, UM-Chor1 cells were killed at very low levels by NK cells regardless of NK phenotype as can be seen from the bar graphs for all allele types in FIG. 5. Panel A. However, cetuximab increased NK-cell lysis in all the NK-cell phenotypes to varying degree: Cetuximab-induced lysis by NK cells from three donors expressing the FcgRIIIa-158 FF was 24%, 17%, and 15%, respectively. Notably, cetuximab-induced ADCC lysis by NK cells using three VF donors was 34%, 49%, and 32%, respectively, and 51%, 66%, and 59% lysis, respectively, using NK cells from three VV donors. As can be seen from Panel B, there was a significant positive correlation (R²=0.85) for the mean of cetuximab-mediated ADCC lysis induced by NK cells from three FF (19%), three VF (38%), three VV (59%) donors. Taken together, these results demonstrate that NK cells that express the FcgRIIIa-158 V allotype (as haNK cells express as well) exhibit significantly enhanced cetuximab-mediated ADCC in chordoma cells.

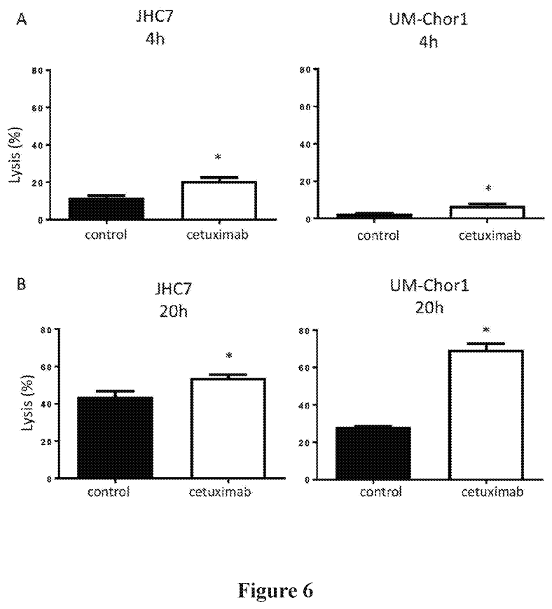

To examine the potential utility of haNK cells for cetuximab therapy of chordoma, the inventor performed an in vitro assay for cetuximab-mediated ADCC using haNK cells as effectors (FIG. 6A). Lysis by haNK cells with isotype control was 11.8% of JHC7 cells and 2.6% of UM-Chor1 cells. Cetuximab significantly enhanced haNK-cell lysis compared to isotype control in both JHC7 (1.7-fold; P<0.01) and UM-Chor1 cells (2.6-fold; P<0.01). The addition of CD16 neutralizing antibody inhibited cetuximab-enhanced haNK-cell lysis in both JHC7 and UM-Chor1 cell lines (data not shown). As NK cells have previously been shown to be “serial killers” (one NK cell can lyse up to five target cells), 20-h ¹¹¹In-release assays were also carried out (FIG. 6B). ADCC assays were performed using two chordoma cell lines, using haNK cells as effector cells at an E:T ratio of 20:1 for A. 4 h and B. 20 h. Indicated groups were incubated with cetuximab and/or anti-CD16 antibody. Statistical analyses were done by Student's t-test, *=P<0.05, error bars indicate mean±S.D. for triplicate measurements. This experiment was repeated at least two times with similar results.

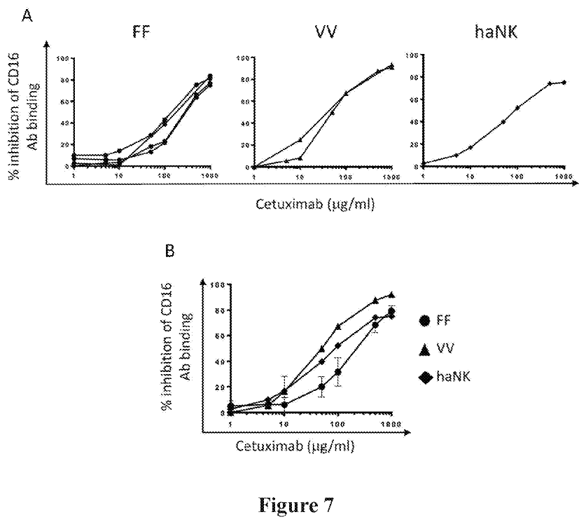

Here, the lysis of the two chordoma cell lines was markedly greater after 20 hours as compared to the 4 hour data in Panel A. These results indicate that haNK cells induce persistent ADCC via cetuximab in chordoma cells. To determine relative affinities, the inventor compared the ability of cetuximab to inhibit the binding of FITC-conjugated CD16 MAb to CD16 polymorphism-genotyped normal donor NK cells and haNK cells (FIG. 7A). Remarkably, a 50% inhibition of CD16 Ab binding to NK cells from four FF donors was achieved with 220 μg/mL of cetuximab. Compared to FF donors, a 4.5-fold lower (49.2 μg/mL) and 2.8-fold lower (80 μg/mL) concentration of cetuximab showed a 50% inhibition of CD16 Ab binding to normal NK cells from VV donor and haNK cells, respectively (FIG. 7B). These results show that both NK cells expressing FcgRIIIa-158 VV and haNK cells bind cetuximab with higher affinity than NK cells expressing FcgRIIIa-158 FF. More specifically, NK cells from four FF and two VV normal donors and haNK cells (NantKwest. 9920 Jefferson Blvd., Culver City, Calif. 90232) were incubated with varying concentrations of cetuximab, followed by FITC-conjugated CD16 Ab. Percentages of inhibition of CD16 MAb binding were calculated as described above Panel A depicts percentages of inhibition of CD16 MAb binding shown by each donor. Panel B depicts the mean of percentages of inhibition of CD16 MAb binding.

In Vivo Examples

In view of the above, numerous treatment protocols in vivo (typically in human) are contemplated that will preferably also include additional treatment regimens or modalities that will complement the targeted immune therapy using hanK cells and cetuximab (or other targeting antibody).

For example, one contemplated treatment will be administered in two phases, an induction and a maintenance phase, as described in more detail below. Preferably, patients will receive induction treatment for up to 1 year. Patients with complete response (CR) in the induction phase, ongoing stable disease (SD) or an ongoing partial response (PR) at 1 year will then proceed to the maintenance phase, and patients will remain in the maintenance phase for up to 1 year.

Tumors will be assessed at screening, and tumor response will be assessed every 8 weeks in the first year or until complete response, and every 12 weeks in the second year or after a complete response by computed tomography (CT) or magnetic resonance imaging (MRI) of target and non-target lesions in accordance with Response Evaluation Criteria in Solid Tumors (RECIST) Version 1.1. For all patients, exploratory tumor molecular profiling will be conducted on samples collected during various time points (e.g., prior to treatment, 8 weeks after the start of treatment, and during potential prolonged treatment periods (depending on response). Separate blood tubes will be collected every 6 weeks in the first year or until a complete response and every 8 weeks in the second year or after a complete response during routine blood draws for immunology and ctDNA/ctRNA analyses.

Contemplated exemplary treatment regimes will include a combination of a vaccine component, low dose metronomic chemotherapy (LDMC), cetuximab, NK cell therapy, low-dose radiation therapy, an IL-15 superagonist, and a checkpoint inhibitor to so maximize immunogenic cell death (ICD) and to augment and maintain the innate and adaptive immune responses against cancer cells. More specifically, the treatment is designed to interrupt the escape phase of immunoediting by: (a) Mitigating potential immunosuppression in the tumor microenvoronment (TME), preferably by LDMC to reduce the density of Tregs, MDSCs, and M2 macrophages that contribute to immunosuppression in the TME; (b) Inducing and coordinating ICD signals, preferably via LDMC and low-dose radiation therapy to increase the antigenicity of tumor cells. Cetuximab and avelumab will be used to enhance ADCC and cytotoxic T-cell activity; (c) Conditioning dendritic and T cells, preferably by cancer vaccines and an IL-15 superagonist to enhance tumor-specific cytotoxic T-cell responses; (d) Enhancing innate immune responses, preferably using NK cell therapy (e.g., in combination with cetuximab) will be used to augment the innate immune system, and an IL-15 superagonist will be used to enhance the activity of endogenous and introduced NK cells. (e) Hypofractionated-dose radiation therapy to upregulate tumor cell NK ligands to enhance tumor cytotoxicity of NK cells; and maintaining immune responses. Checkpoint inhibitors will be used to promote long-term anticancer immune responses.

To that effect, suitable agents included in the exemplary treatment are summarized in Table 1. It should therefore be recognized that by combining the agents that simultaneously target distinct but complementary mechanisms that enable tumor growth, the treatment regimen aims to maximize anticancer activity and prolong the duration of response to treatment. Moreover, the treatment will typically be administered in 2 phases: an induction phase and a maintenance phase. The purpose of the induction phase is to stimulate immune responses against tumor cells and mitigate immunosuppression in the TME. The purpose of the maintenance phase is to sustain ongoing immune system activity against tumor cells, creating durable treatment responses.

TABLE 1

| | Mitigating | | | Enhancing | |
|---|---|---|---|---|---|
| | immuno- | inducing and | Conditioning | Innate | Maintaining |
| | suppression | Coordinating | Dendritic | Immune | Immune |
| Agent | in the TME | ICD Signals | and T Cells | Responses | Responses |
| Aldoxorubicin HCl | X | X | | | |
| ALT-803 | | | X | X | X |
| Ad5-based vaccines: | | | X | | |
| ETBX-051 (Brachyury) | | | | | |
| and ETBX-061 (MUC1) | | | | | |
| Yeast-based vaccine: | | | X | | |
| GI-6301 (Brachyury) | | | | | |
| haNK cells | | | | X | |
| Avelumab | | | | | X |
| Cetuximab | | X | | | |
| Cyclophosphamide | X | X | | | |
| SBRT | | X | | X | |

Aldoxorubicin hydrochloride (HCl): Aldoxorubicin HCl is an albumin-binding prodrug of the anticancer agent doxorubicin. Due to enhanced permeability of the vasculature within tumors, plasma albumin preferentially accumulates in solid tumors. Aldoxorubicin HCl binds circulating albumin through a thiol reactive maleimide group conjugated to the doxorubicin molecule; binding to albumin results in targeting and accumulation of the aldoxorubicin HCl prodrug in solid tumors. Doxorubicin has been postulated to act through a number of mechanisms including intercalation of DNA, inhibition of topoisomerase II, induction of apoptosis, inhibition of RNA synthesis, and/or interaction with the cell membrane. The chemical name for aldoxorubicin HCl is N-[(E)-[1-[(2S,4S)-4-[(2R,4S,5S,6S)-4-amino-5-hydroxy-6-methyloxan-2-yl]oxy-2,5,12-trihydroxy-7-methoxy-6,11-dioxo-3,4-dihydro-1H-tetracen-2-yl]-2-hydroxyethylidene]amino]-6-(2,5-dioxopyrrol-1-yl)hexanamide; hydrochloride. Aldoxorubicin is manufactured by Baxter Oncology.

ALT-803 (recombinant human super agonist interleukin-15 (IL-15) complex [also known as IL15N72D:IL-15RαSu/IgG1 Fc complex]): ALT-803 is an IL-15-based immunostimulatory protein complex consisting of two protein subunits of a human IL-15 variant associated with high affinity to a dimeric human IL-15 receptor a (IL-15Rα) sushi domain/human IgG1 Fc fusion protein. The IL-15 variant is a 114 amino acid polypeptide comprising the mature human IL-15 cytokine sequence, with an asparagine to aspartate substitution at position 72 of helix C (N72D). The human IL-15Rα sushi domain/human IgG1 Fc fusion protein comprises the sushi domain of the human IL-15 receptor a subunit (IL-15Rα) (amino acids 1-65 of the mature human IL-15Rα protein) linked to the human IgG1 CH2-CH3 region containing the Fc domain (232 amino acids). Except for the N72D substitution, all of the protein sequences are human. ALT-803 is manufactured by Altor Biosciences.

ETBX-051 (Ad5 [E1-, E2b-]-Brachyury vaccine): ETBX-051 is an Ad5-based vector that has been modified by the removal of the E1, E2b, and E3 gene regions and the insertion of a modified hBrachyury gene. The modified hBrachyury gene contains agonist epitopes designed to increase cytotoxic T-lymphocyte (CTL) antitumor immune responses. ETBX-051 is manufactured by Etubics.

ETBX-061 (Ad5 [E1-, E2b-]-mucin 1 [MUC1] vaccine): ETBX-061 is an Ad5-based vector that has been modified by the removal of the E1, E2b, and E3 gene regions and the insertion of a modified human MUC1 gene. The modified MUC1 gene contains agonist epitopes designed to increase CTL antitumor immune responses. ETBX-061 is manufactured by Etubics.

GI-6301 (Brachyury yeast vaccine): GI-6301 is a heat-killed S. cerevisiae yeast-based vaccine expressing the hBrachyury oncoprotein. The Brachyury antigen is the full-length protein possessing an N-terminal MADEAP (Met-Ala-Asp-Glu-Ala-Pro) motif appended to the hBrachyury sequence to promote antigen accumulation within the vector and a C-terminal hexahistidine epitope tag for analysis by Western blotting. Expression of the hBrachyury protein is controlled by a copper-inducible CUP1 promoter. GI-6301 is manufactured by Globelmmune.

haNK™, NK-92 [CD16.158V, ER IL-2], Suspension for Infusion (haNK™ for Infusion): NK-92 [CD16.158V, ER IL-2] (high-affinity activated natural killer cell line, [haNK™ for Infusion]) is a human, allogeneic, NK cell line that has been engineered to produce endogenous, intracellularly retained IL-2 and to express CD16, the high-affinity (158V) Fc gamma receptor (FcγRIIIa/CD16a). Phenotypically, the haNK cell line is CD56+, CD3−, and CD16+.

The haNK cell line was developed by transfecting the parental activated NK (aNK) cell line (NK-92) with a bicistronic plasmid vector containing IL-2 and the high-affinity variant of the CD16 receptor. The plasmid contains an ampicillin resistance cassette, and the promoter used for expression of the transgene is elongation factor 1 alpha with an SV40 polyadenylation sequence. The plasmid was made under transmissible spongiform encephalopathies-free production conditions and contains some human origin sequences for CD16 and IL-2, neither of which have any transforming properties. haNK™ for Infusion has enhanced CD16-targeted ADCC capabilities as a result of the insertion of the high-affinity variant of the CD16 receptor. haNK cells are manufactured by NantKwest.

Avelumab (commercially available from Pfizer as BAVENCIO® injection, for intravenous [IV] use): Avelumab is a human IgG1 lambda monoclonal antibody directed against the human immunosuppressive PD-L 1 protein and has potential immune checkpoint inhibitory and antineoplastic activities. Avelumab has a molecular weight of 147 kDa. By inhibiting PD-L1 interactions, avelumab is thought to enable the activation of T cells and the adaptive immune system. By retaining a native Fc-region, avelumab is thought to engage the innate immune system and induce ADCC.

Cetuximab (commercially available from Eli Lilly as ERBITUX® injection, for IV infusion): Cetuximab is a recombinant, human/mouse chimeric monoclonal antibody that binds specifically to the extracellular domain of human EGFR. Cetuximab is composed of the Fv regions of a murine anti-EGFR antibody with human IgG1 heavy and kappa light chain constant regions and has an approximate molecular weight of 152 kDa. Cetuximab is produced in mammalian (murine myeloma) cell culture. Cetuximab is a sterile, clear, colorless liquid of pH 7.0 to 7.4, which may contain a small amount of easily visible, white, amorphous cetuximab particulates. Cetuximab is supplied at a concentration of 2 mg/mL in either 100 mg (50 mL) or 200 mg (100 mL), single-use vials. Cetuximab is formulated in a solution with no preservatives, which contains 8.48 mg/mL sodium chloride, 1.88 mg/mL sodium phosphate dibasic heptahydrate, 0.41 mg/mL sodium phosphate monobasic monohydrate, and Water for Injection, USP.

Cyclophosphamide (commercially available as Cyclophosphamide Capsules, for oral use; or Cyclophosphamide Tablets, USP): Cyclophosphamide is a synthetic antineoplastic drug chemically related to the nitrogen mustards. The chemical name for cyclophosphamide is 2-[bis(2-chloroethyl)amino]tetrahydro-2H-1,3,2-oxazaphosphorine 2-oxide monohydrate and has the molecular formula C7H15C12N2O2P.H2O and a molecular weight of 279.1. Each capsule for oral use contains 25 mg or 50 mg cyclophosphamide (anhydrous, USP).

Stereotactic body radiation therapy (SBRT): SBRT has emerged as a safe and effective alternative to conventionally-fractionated external beam radiation. SBRT is a highly conformal external beam radiation technique, capable of precisely delivering ablative doses of radiation in a limited number of fractions. Preclinical data suggest relatively large doses per fraction (6-8 Gy) can induce immune responses to tumor antigens. The steep dose fall-off observed with SBRT treatments allows high doses per fraction to be achieved with limited radiation exposure to adjacent critical structures.

Most typically patients will receive 4 fractions of radiation per feasible tumor site (maximum of 5 sites), at a dose of up to 8 Gy per fraction. If organ at risk (OAR) dose constraints cannot be achieved, a dose reduction to 6 Gy per fraction can be performed at the discretion of the treating physician. Radiation treatments will be administered twice every 21 days for the first 2 treatment cycles. A single treatment plan will be devised for each lesion prior to initiation of therapy. Given the length of time between fractions, a repeat CT simulation and adjustments to the treatment plan may be performed at the discretion of the radiation oncologist if significant tumor regression (as noted radiographically or by clinical exam) occurs between fractions. Changes to the treatment plan should only be made to exclude normal tissues or critical structures that are clearly uninvolved by tumor and which may have fallen into the GTV as a result of tumor regression.

Radiation dose will be prescribed such that 95% of the PTV receives the prescription dose or greater, though reductions to as low as 80% coverage will be considered acceptable if deemed appropriate by the treating physician in order to spare critical normal structures; in such cases, the region receiving less than 95% of the prescription dose should be limited to the periphery of the PTV and outside of the GTV. A high degree of dose heterogeneity is to be expected with SBRT. As such a central “hotspot” is expected, and the prescription dose should be within 60 90% of the maximum dose within the PTV. Radiation dose calculations will be performed using tissue heterogeneity corrections

While not limiting to the inventive subject matter, contemplated pharmaceutical agents and radiation will be administered following the exemplary dosages listed in Table 2. Of course, it should be appreciated that patient and disease specific factors (e.g., gender, weight, disease response or progression, adverse reactions, etc.) may dictate a change in the particular dosage and schedule.

TABLE 2

| | | | Mode of |
|---|---|---|---|
| Drugs | Dosage | | Administration |
| Aldoxorubicin HCl | 80 | mg/m² | IV over |
| | | | approximately 30 |
| | | | minutes |
| ALT-803 | 10 | μg/kg | SC |
| Ad5-based vaccines: ETBX- | 1 × 10¹¹ | | SC |
| 051 (Brachyury) and ETBX- | VP/vaccine/dose | | |
| 061 (MUC1) | | | |
| Yeast-based vaccine: | 80 | YU/dose | SC |
| GI-6301 (Brachyury) | | | |
| haNK | 2 × 10⁹ | | IV |
| | cells/dose | | |
| Avelumab | 10 | mg/kg | IV |
| Cetuximab | 250 | mg/m² | IV |
| Cyclophosphamide | 25 | mg BID | PO |
| | (days 1-5) | | |
| | 25 | mg daily | |

| | | |
|---|---|---|
| | (days 8-12) | |
| SBRT | 8 Gy maximum | External beam |
| | (exact dose to be | radiation |
| | determined by the | |
| | radiation oncologist) | |

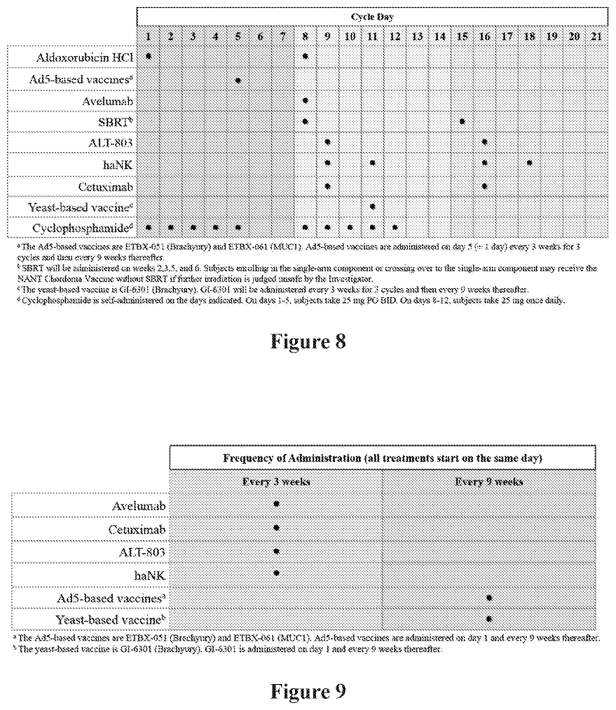

A typical treatment schema for the induction phase is shown in FIG. 8, and a typical treatment schema for the maintenance phase is shown in FIG. 9.

For example, an exemplary treatment regimen for the induction phase is contemplated, lasting about 8 weeks (minimum) to about 1 year (maximum). Treatment will include repeated 3-week cycles for a maximum treatment period of 2 years, as follows:

Days 1 and 8, every 3 weeks: Aldoxorubicin HCl (80 mg/m2 IV over approximately 30 minutes).

Days 1-5, every 3 weeks: Cyclophosphamide (25 mg by mouth [PO] twice a day [BID]).

Day 5 (±1 day), every 3 weeks for 3 cycles then every 9 weeks thereafter: Ad5-based vaccines: ETBX-051 (Brachyury) and ETBX-061 (MUC1), (1×10¹¹ virus particles [VP]/vaccine/dose subcutaneously [SC]).

Day 8, every 3 weeks: Avelumab (10 mg/kg IV over approximately 1 hour).

Days 8-12, every 3 weeks: Cyclophosphamide (25 mg by mouth [PO] daily).

Days 8 and 15, every 3 weeks: SBRT (not to exceed 8 Gy, exact dose to be determined by the radiation oncologist: for the first 2 cycles only).

Day 9, every 3 weeks: ALT-803 (10 μg/kg SC at least 30 minutes prior to haNK infusion); haNK (2×10⁹ cells/dose IV); Cetuximab (250 mg/m² IV).

Days 11, every 3 weeks: haNK (2×10⁹ cells/dose IV).

Day 11, every 3 weeks for 3 cycles and every 9 weeks thereafter: Yeast-based vaccine: GI-6301(Brachyury) (80 yeast units [YU]/dose SC).

Day 16, every 3 weeks: ALT-803 (10 μg/kg SC at least 30 minutes prior to haNK infusion); haNK (2×10⁹ cells/dose IV); Cetuximab (250 mg/m² IV).

Day 18, every 3 weeks: haNK (2×10⁹ cells/dose IV).

An exemplary treatment regimen for the maintenance phase, which may last up to 1 year following completion of the last treatment in the induction phase will include repeated cycles, as follows:

Day 1, every 3 weeks: Avelumab (10 mg/kg IV over approximately 1 hour); Cetuximab (250 mg/m² IV); ALT-803 (10 μg/kg SC) (at least 30 minutes prior to haNK infusion); haNK (2×10⁹ cells/dose IV).

Day 1, every 9 weeks: Ad5-based vaccines: ETBX-051 (Brachyury) and ETBX-061 (MUC1) (1×10¹¹ VP/vaccine/dose SC); Yeast-based vaccine: GI-6301 (Brachyury) (80 YU/dose SC), approximately 2 hours after administration of Ad-5 based vaccines.

For tumor response evaluation it is contemplated that patients will be evaluated for tumor burden by CT and/or MRI imaging at screening (up to 28 days before treatment). Subsequent evaluations for tumor response will occur every 8 weeks or 12 weeks (depending on time on treatment, as described previously) (±7 days) following the administration of the first treatment. Imaging will continue until PD is documented or the subject completes study follow-up. When disease progression per RECIST Version 1.1 is initially observed, an imaging assessment will be done 4-6 weeks after the initial PD assessment to rule out tumor pseudoprogression. For patients exhibiting a response (PR or CR), a confirmatory imaging assessment will be done 4-6 weeks after the initial response. Evaluations may include CT and/or MRI scans of the chest, abdomen, pelvis (optional unless known pelvic disease is present at baseline), and brain (only as clinically warranted based on symptoms/findings).

Prior to treatment, tumor lesions to be followed for response will be clearly identified by location and selected and categorized as target or non-target lesions. Target lesions include those lesions that can be accurately measured in at least 1 dimension as ≥10 mm, using CT, PET-CT, or MRI with a slice thickness ≤5 mm. Malignant lymph nodes with a short axis diameter ≥15 mm can be considered target lesions. Up to a maximum of 2 target lesions per organ and 5 target lesions in total will be identified at baseline. These lesions should be representative of all involved organs and selected based on their size (those with the longest diameter) and their suitability for accurate repeated measurements. A sum of the longest lesion diameter (LLD) for all target lesions will be calculated and reported as the baseline sum LLD. For malignant lymph nodes identified as target lesions, the short axis diameter will be used in the sum of LLD calculation. All other lesions (or sites of disease) should be identified as non target lesions (including bone lesions).

All post-baseline response assessments should follow the same lesions identified at baseline. The same mode(s) of assessment (e.g., CT or MRI) used to identify/evaluate lesions at baseline should be used throughout the course of the study unless subject safety necessitates a change (e.g., allergic reaction to contrast media).

For tumor molecular profiling it is contemplated that genomic sequencing of tumor cells from tissue relative to non-tumor cells from whole blood will be conducted to identify tumor-specific genomic variances that may contribute to disease progression and/or response to treatment. RNA sequencing will be conducted to provide expression data and give relevance to DNA mutations. Quantitative proteomics analysis will be conducted to determine the absolute amounts of specific proteins, to confirm expression of genes that are correlative of disease progression and/or response, and to determine cutoff values for response.

Tumor molecular profiling will preferably be performed on FFPE tumor tissue and whole blood (subject-matched normal comparator against the tumor tissue) by next-generation sequencing and mass spectrometry-based quantitative proteomics. Tumor tissue from a biopsy will also be collected 8 weeks after the start of treatment. Furthermore, if additional tumor biopsies will be performed, further tumor molecular profiling will be performed on those samples, as well.

For example, tumor tissue and whole blood samples will be collected and shipped in accordance with the instruction cards included in a Tissue Specimen Kit and Blood Specimen Kit. An FFPE tumor tissue specimen is typically used for the extraction of tumor DNA, tumor RNA, and tumor protein. A whole blood sample is typically used for the extraction of subject normal DNA. Tumor tissue and whole blood will be processed in a CLIA certified and CAP-accredited clinical laboratories (e.g., NantOmics, LLC; ResearchDx. LLC; and Expression Pathology, Inc. dba OncoPlex Diagnostics).

Immunology Analysis: Whole blood for immunology analysis will be collected, every 6 weeks in the induction phase and every 8 weeks in the maintenance phase during routine blood draws, and at the end of treatment. If a tumor biopsy will be performed at screening, blood samples for immunology analysis may be collected prior to the biopsy. Blood samples will be stored in a laboratory to be determined. Immune responses will be evaluated by standard immune assays. Correlations between therapy-induced immune changes and subject outcomes will be assessed.

Circulating Tumor DNA and RNA Assays: Tumors evolve during therapy, and drug-resistant cells emerge, which are difficult to detect and may cause the tumor to become resistant to the initial treatment. Blood-based testing for ctDNA and ctRNA can track the emergence of drug-resistant tumor cells and can identify new drug targets and treatment options for patients. To that end, whole blood for ctDNA/ctRNA analysis will be collected during the screening period for subjects who have been enrolled in the study, every 6 weeks in the induction phase and every 8 weeks in the maintenance during routine blood draws, and at the end of treatment. If a tumor biopsy will be performed at screening, blood samples for ctDNA and ctRNA analysis must be collected prior to the biopsy. Expression levels of specific tumor- and immune-related analytes in ctDNA and ctRNA will be measured by qPCR and possibly other methods (e.g., DNA/RNA sequencing) and analyzed for correlations with subject outcomes.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Furthermore, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of treating chordoma, comprising:
co-administering an anti-EGFR antibody and a high affinity NK (haNK) cell to a patient in need thereof at a dosage effective to treat the chordoma.

2. The method of claim 1 wherein the anti-EGFR antibody is a monoclonal antibody with binding specificity against human EGFR.

3-14. (canceled)

15. The method of claim 1 wherein the further cancer treatment comprises an immune therapy.

16. The method of claim 15 wherein the immune therapy comprises administration of a recombinant yeast or recombinant virus expressing a patient- and tumor-specific neoepitope.

17. The method of claim 15 wherein the immune therapy comprises administration of a recombinant yeast or recombinant virus expressing brachyury.

18. The method of claim 1 wherein the further cancer treatment comprises a chemotherapy.

19. The method of claim 1 wherein the chemotherapy comprises administration of at least one of aldoxorubicin, cyclophosphamide, irinotecan, gemcitabine, capecitabine, 5-FU, FOLFIRI, FOLFOX, and oxiplatin.

20. The method of claim 1 wherein the further cancer treatment comprises a radiotherapy.

21. The method of claim 1 wherein the anti-EGFR antibody is a monoclonal antibody with binding specificity against human EGFR.

22. The method of claim 1 wherein the anti-EGFR antibody is an IgG1.

23. The method of claim 1 wherein the anti-EGFR antibody is a humanized non-human anti-EGFR antibody.

24. The method of claim 1 wherein the anti-EGFR antibody is cetuximab.

25. The method of claim 1 wherein the anti-EGFR antibody is administered at a dosage of between 100 mg/m2 and 1,000 mg/m2.

26. The method of claim 1 wherein the anti-EGFR antibody is co-administered at the same time as the haNK cell.

27. The method of claim 1 wherein the anti-EGFR antibody is bound to a high-affinity CD16 that is expressed on a surface of the haNK cell.

28. The method of claim 1 wherein the haNK cell is administered at a dosage of between 5×10⁵ cells/kg and 5×10⁸ cells/kg.

29. The method of claim 1 wherein the haNK cell is a NK92 derivative that further express recombinant IL2.

30. (canceled)

31. The method of claim 1 wherein the haNK cell is genetically engineered to have a reduced expression of at least one inhibitory receptor.

32. The method of claim 1 wherein the haNK cell is irradiated before administration at a radiation dose of at least 500 cGy.

33. The method of claim 1 further comprising a step of administering a further cancer treatment to the patient.

34. The method of claim 33 wherein the further cancer treatment comprises an immune therapy.

35. The method of claim 34 wherein the immune therapy comprises administration of a recombinant yeast or recombinant virus expressing a patient- and tumor-specific neoepitope.

36. The method of claim 34 wherein the immune therapy comprises administration of a recombinant yeast or recombinant virus expressing brachyury.

37. The method of claim 33 wherein the further cancer treatment comprises a chemotherapy or radiotherapy.

38. The method of claim 37 wherein the chemotherapy comprises administration of at least one of irinotecan, gemcitabine, capecitabine, 5-FU, FOLFIRI, FOLFOX, and oxiplatin.

39. The method of claim 33 wherein the further cancer treatment comprises a radiotherapy.

40. A pharmaceutical composition comprising an anti-EGFR antibody and a genetically engineered NK cell, wherein a high affinity variant of CD16 is expressed on a surface of the genetically engineered NK cell, and wherein the anti-EGFR antibody is optionally bound to the high affinity variant of CD16 of the genetically engineered NK cell.

41. The pharmaceutical composition of claim 40 wherein the antibody is a monoclonal antibody.

42-43. (canceled)

44. The pharmaceutical composition of claim 40 wherein the antibody is cetuximab.

45-49. (canceled)

50. The pharmaceutical composition of claim 40 wherein the antibody is a monoclonal antibody.

51-54. (canceled)

55. The pharmaceutical composition of claim 40 wherein the genetically engineered NK cell further expresses recombinant IL2.

56. The pharmaceutical composition of claim 40 wherein the genetically engineered NK cell is genetically engineered to have a reduced expression of at least one inhibitory receptor.

57. (canceled)

58. The pharmaceutical composition of claim 40 wherein the composition is formulated for transfusion and comprises between 1×10⁶ cells and 5×10⁹ cells.

59-75. (canceled)
